(19) United States
(12) Patent Application Publication
(10) Pub. No.: US 2025/0281092 A1
(43) Pub. Date: Sep. 11, 2025

(54) HEAD-WORN DEVICE

(71) Applicant: Shenzhen PanBrain Technology Co., Ltd., Shenzhen (CN)

(72) Inventor: Nailin YAO, Shenzhen (CN)

(73) Assignee: Shenzhen PanBrain Technology Co., Ltd., Shenzhen (CN)

(21) Appl. No.: 19/073,589

(22) Filed: Mar. 7, 2025

(30) Foreign Application Priority Data
Mar. 8, 2024 (CN) 202420473127.4

(51) Int. Cl. A61B 5/291; A61B 5/256

(52) U.S. Cl. CPC A61B 5/291; A61B 5/256

(57) ABSTRACT

A head-worn device is arranged with a hollow shell, a first connecting member, a second connecting member, a knob, and a headband. An accommodating space is formed in the hollow shell, and two through holes are arranged on the hollow shell. A mounting hole is arranged on the hollow shell. The mounting hole and the through holes are all connected to the accommodating space. The two connecting members are arranged in the accommodating space, and are able to reciprocate along an axial direction of the accommodating space. The knob is inserted into the mounting hole and extends between the two connecting members. Two sides of the knob are respectively connected to the two connecting members. The headband is arranged on the hollow shell, two ends of the headband respectively pass through the two through holes and are respectively connected to the two connecting members.

### CROSS-REFERENCE TO RELATED APPLICATIONS

The present disclosure claims priority to Chinese Patent Application No. 202420473127.4, filed on Mar. 8, 2024, the content of all of which is incorporated herein by reference.

### FIELD

The present disclosure relates to the technical field of wearable devices, in particular to a head-worn device.

### BACKGROUND

With the development of technology, an electroencephalogram is a device used to measure and record human brain activity. The electroencephalogram typically consists of multiple sensors (electrodes) placed on the scalp to capture electrical signals emitted by the brain. These signals are amplified, filtered, and digitized for analysis and interpretation. During the use of a head-mounted electroencephalogram, the device is often worn around the head. However, due to variations in users' head shapes, facial structures, and body sizes, the tightness of the device may differ among different users.

Accordingly, a reliable and user-friendly adjustment mechanism is needed to regulate the tightness of the head-worn device. A common adjustment method involves the use of an elastic band. For example, Chinese Patent Publication No. CN116036474A discloses a wearable transcutaneous acupoint electrical stimulation therapy device for preventing and treating postoperative cognitive dysfunction. The specification of the patent describes that the therapy device utilizes an adjustable elastic band along the coronal plane of the head to accommodate different head circumferences and securely fasten the wearable device to the patient's head.

However, elastic band generally provides only limited adjustability in terms of tightness and tends to lose elasticity over prolonged use, making effective adjustment of tightness difficult.

Therefore, the prior art still needs to be improved and developed.

### SUMMARY

In view of the above-mentioned deficiencies in the prior art, an object of the present disclosure is to provide a head-worn device, aiming to solve the problem that the elastic band can only provide limited tightness adjustment and loses elasticity during prolonged use, resulting in difficulties of effective adjustment of tightness.

The technical solution of the present disclosure is as follows:

A head-worn device includes a hollow shell, a first connecting member, a second connecting member, a knob, and a headband, an accommodating space is formed in the hollow shell, and two through holes are oppositely arranged on an inner side wall of the hollow shell; a mounting hole is arranged on an outer side wall of the hollow shell; the mounting hole and the through holes are all connected to the accommodating space; the first connecting member and the second connecting member are arranged in parallel in the accommodating space, and the first connecting member and the second connecting member are both able to reciprocate along an axial direction of the accommodating space; the knob is inserted into the mounting hole and extends between the first connecting member and the second connecting member; two sides of the knob are respectively connected to the first connecting member and the second connecting member, and are used to drive the first connecting member and the second connecting member to move toward or away from each other; and the headband is arranged on a side wall of the hollow shell; two ends of the headband respectively pass through the two through holes and are respectively connected to the first connecting member and the second connecting member.

In the head-worn device, a first strip-shaped through hole is arranged on the first connecting member, and a first rack is arranged on a side wall of the first strip-shaped through hole along a length direction; a second strip-shaped through hole is arranged on the second connecting member, and a second rack is arranged on a side wall of the second strip-shaped through hole along a length direction; the first rack and the second rack are arranged opposite to each other, and the knob includes an adjusting gear, the adjusting gear is rotatably arranged in the first strip-shaped through hole and the second strip-shaped through hole, and a gear ring portion is arranged on a circumference of the adjusting gear, and the first rack and the second rack are respectively meshed with opposite sides of the gear ring portion.

In the head-worn device, a ratchet ring is arranged on a side wall of the mounting hole, and the knob includes a mounting cover and a stopping body; the mounting cover is arranged on one side of the mounting hole away from the accommodating space, and a central shaft is arranged on the mounting cover, and the adjusting gear is sleeved on the central shaft, the mounting cover is used to drive the adjusting gear to rotate; the stopping body is sleeved on the central shaft and rotates synchronously with the adjusting gear, a pawl portion is arranged on an outer peripheral wall of the stopping body, and the pawl portion includes a stopping surface and a guiding inclined surface, and when the stopping body rotates in a direction toward the stopping surface, the stopping surface engages with the ratchet ring; when the stopping body rotates in a direction toward the guiding inclined surface, the guiding inclined surface and the ratchet ring slide relative to each other.

In the head-worn device, an angle between the guiding inclined surface and the stopping body is an acute angle; and an angle between the stopping surface and the stopping body is a right angle or an obtuse angle.

In the head-worn device, a rib column is arranged on a side of the mounting cover facing the accommodating space, and an arc groove adapted to the rib column is arranged on the stopping body, the rib column is inserted into the arc groove, and when the mounting cover rotates, the rib column abuts against the arc groove to push the stopping body to rotate.

In the head-worn device, a push column is further arranged on the side of the mounting cover facing the accommodating space, a hook opening is arranged on the stopping body, an end of the stopping body is bent toward the hook opening to form a hook-shaped part, and the push column is arranged to pass through the hook opening, when the stopping body rotates in a direction toward the stopping surface, the push column pushes the hook-shaped part inward to disengage the stopping surface and the ratchet ring.

In the head-worn device, the stopping body and the adjusting gear are integrally formed; and/or the stopping body and the pawl portion are integrally formed.

In the head-worn device, at least two pawl portions are arranged at intervals along a circumferential direction on an outer edge of the ratchet ring.

In the head-worn device, the hollow shell includes a wearable shell and an assembly shell, the assembly shell cooperates with the wearable shell to form the accommodating space, the headband is arranged on the wearable shell, and the mounting hole is opened on the assembly shell; a first guiding column and a second guiding column are arranged on a side of the assembly shell facing the accommodating space, the first guiding column and the second guiding column are arranged at intervals along a width direction of the assembly shell, and the first connecting member or the second connecting member is arranged between the first guiding column and the second guiding column.

In the head-worn device, a first positioning groove is arranged on the first guiding column, a first positioning column adapted to the first positioning groove is arranged on the wearable shell, and the first positioning column is inserted into the first positioning groove; and/or a second positioning groove is arranged on the second guiding column, a second positioning column adapted to the second positioning groove is arranged on the wearable shell, and the second positioning column is inserted into the second positioning groove.

Compared with the prior art, the embodiment of the present disclosure has the following advantages:

The present disclosure provides a head-worn device with adjustable tightness and easy operation, which is arranged with a hollow shell for wearing on the top of the user's head, and is also arranged with a first connecting member, a second connecting member and a headband. The headband passes through through holes and is connected with the first connecting member and the second connecting member, enabling the entire head-mounted device to be securely fastened to the user's head, thereby reducing movement and shaking. The first connecting member and the second connecting member, connected with the headband, form an annular structure that encircles the user's head. The user can adjust the relative movement of the first connecting member and the second connecting member toward or away from each other via a knob, thereby adjusting the length of the overlapping portion between the first connecting member and the second connecting member. The adjustment can be performed without additional tools or operations, allowing for precise tightness adjustment to accommodate users with different head sizes. Furthermore, the adjustment can be performed by touch without requiring visual confirmation, making the operation simple and convenient. The present disclosure reduces sliding and shaking of the head-worn device, providing improved stability.

The head-worn device can still maintain stability and comfort during long-term use. By using the head-worn device, the tightness of the head-worn device can be flexibly adjusted according to the head circumference of different users to obtain better wearing comfort and stability.

### BRIEF DESCRIPTION OF THE DRAWINGS

In order to more clearly illustrate the embodiments of the present disclosure or the technical solutions in the prior art, the drawings required for use in the embodiments are briefly introduced below. Obviously, the drawings described below are only some embodiments recorded in the present disclosure. For those skilled in the art, other drawings can be obtained based on these drawings without involving creative work.

FIG. 1 is an assembly diagram of a head-worn device according to the present disclosure.

FIG. 2 is an exploded view of the structure of the head-worn device according to the present disclosure.

FIG. 3 is another exploded view of the structure of the head-worn device according to the present disclosure.

FIG. 4 is a schematic diagram of the structure of the knob according to the present disclosure.

FIG. 5 is an assembly diagram of the knob, the first connecting member, the second connecting member and the assembly shell according to the present disclosure.

FIG. 6 is an exploded view of the structure of the knob, the first connecting member, the second connecting member and the assembly shell according to the present disclosure.

FIG. 7 is an enlarged view of part A in FIG. 6.

FIG. 8 is a schematic diagram of the structure of the assembly shell and the knob according to the present disclosure.

FIG. 9 is an enlarged view of part B in FIG. 8.

### DETAILED DESCRIPTION OF EMBODIMENTS

In order to enable those skilled in the art to better understand the technical solution of the present disclosure, the drawings of the embodiments of the present disclosure are combined to clearly and completely describe the technical solutions in the embodiments of the present disclosure. Obviously, the described embodiments are only part of the embodiments of the present disclosure, not all of the embodiments. Based on the embodiments of the present disclosure, all other embodiments obtained by those skilled in the art without creative work are within the scope of protection of the present disclosure.

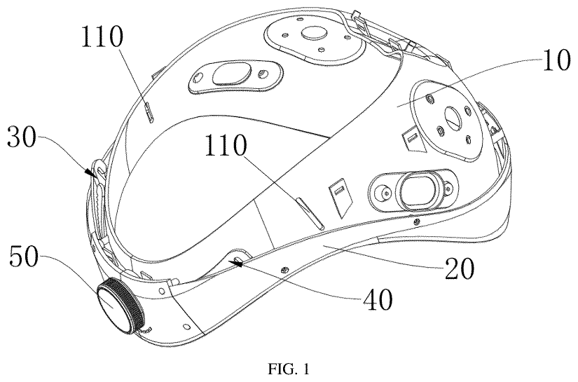

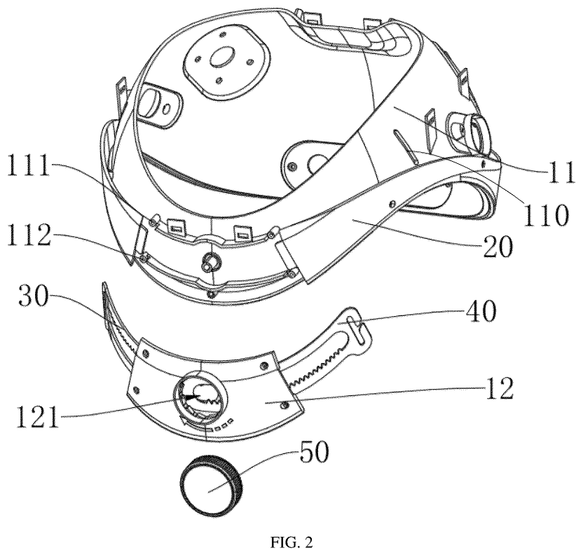

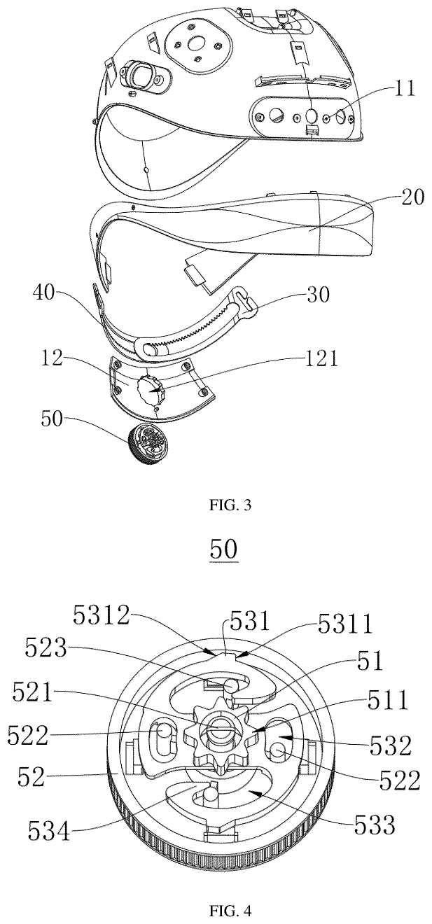

Referring to FIG. 1, FIG. 2 and FIG. 3, in one embodiment of the present disclosure, a head-worn device is disclosed, which includes a hollow shell 10, a first connecting member 30, a second connecting member 40, a knob 50, and a headband 20. An accommodating space is formed in the hollow shell 10, and two through holes 110 are oppositely arranged on an inner side wall of the hollow shell 10. A mounting hole 121 is arranged on an outer side wall of the hollow shell 10; the mounting hole 121 and the through holes 110 are all connected to the accommodating space. The first connecting member 30 and the second connecting member 40 are arranged in parallel in the accommodating space, and the first connecting member 30 and the second connecting member 40 can reciprocate along an axial direction of the accommodating space. The knob 50 is inserted into the mounting hole 121 and extends between the first connecting member 30 and the second connecting member 40. Two sides of the knob 50 are respectively connected to the first connecting member 30 and the second connecting member 40, and are used to drive the first connecting member 30 and the second connecting member 40 to move toward or away from each other. The headband 20 is arranged on a side wall of the hollow shell 10. Two ends of the headband 20 respectively pass through the two through holes 110, and are respectively connected to the first connecting member 30 and the second connecting member 40.

The present embodiment provides a head-worn device with adjustable tightness and easy operation, which is arranged with a hollow shell 10 for wearing on the top of the user's head, and is also arranged with a first connecting member 30, a second connecting member 40 and a headband 20. The headband 20 passes through through holes 110 and is connected with the first connecting member 30 and the second connecting member 40, enabling the entire head-mounted device to be securely fastened to the user's head, thereby reducing movement and shaking. The first connecting member 30 and the second connecting member 40, together with the headband 20, form an annular structure that encircles the user's head. The user can adjust the relative movement of the first connecting member 30 and the second connecting member 40 toward or away from each other via a knob 50, thereby adjusting the length of the overlapping portion between the first connecting member 30 and the second connecting member 40. The adjustment can be performed without additional tools or operations, allowing for precise tightness adjustment to accommodate users with different head sizes. Furthermore, the adjustment can be performed by touch without requiring visual confirmation, making the operation simple and convenient. The present disclosure reduces sliding and shaking of the head-worn device, providing improved stability

Exemplarily, after the user puts on the head-worn device, if the user feels that the head and the head are not firmly fit, the knob 50 can be used to adjust the first connecting member 30 and the second connecting member 40 to move to each other, that is, the length of the overlapping portion between the first connecting member 30 and the second connecting member 40 increases, so that the enclosure structure in contact with the head is tightened as a whole. If the user feels too much pressure on the head after putting on the device, that is, the enclosure is too tight, the knob 50 can be used to adjust the first connecting member 30 and the second connecting member 40 to move away from each other, that is, the length of the overlapping portion between the first connecting member 30 and the second connecting member 40 decreases, so that the enclosure structure in contact with the head becomes loose as a whole and is adjusted to a state suitable for the head circumference.

The disclosed head-worn device can still maintain stability and comfort during long-term use. By using the disclosed head-worn device, the tightness of the head-worn device can be flexibly adjusted according to the head circumference of different users to obtain better wearing comfort and stability.

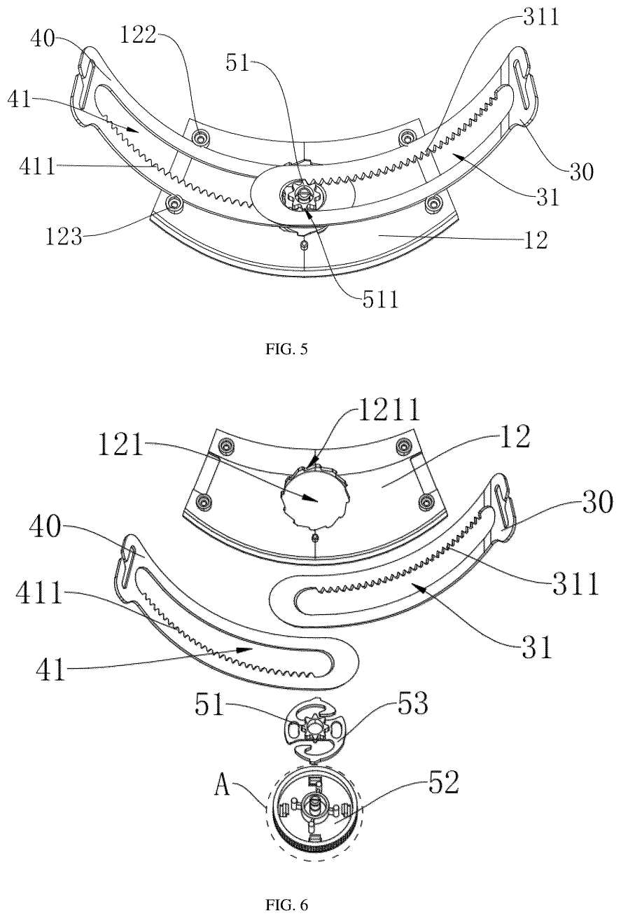

As shown in FIG. 5 and FIG. 6, as another implementation of the present embodiment, the first connecting member 30 is disclosed to be arranged with a first strip-shaped through hole 31, and a first rack 311 is arranged on a side wall of the first strip-shaped through hole 31 along a length direction. The second connecting member 40 is arranged with a second strip-shaped through hole 41, and a second rack 411 is arranged on a side wall of the second strip-shaped through hole 41 along a length direction, the first rack 311 and the second rack 411 are arranged opposite to each other. The knob 50 includes an adjusting gear 51, and the adjusting gear 51 is rotatably arranged in the first strip-shaped through hole 31 and the second strip-shaped through hole 41. A gear ring portion 511 is arranged on a circumference of the adjusting gear 51, the first rack 311 and the second rack 411 are respectively meshed with opposite sides of the gear ring portion 511. The adjusting gear 51 is used to drive the first connecting member 30 and the second connecting member 40 move toward or away from each other, and the tightness of the head circumference can be adjusted by rotating the adjusting gear 51, which is easy to operate and convenient and quick.

The length of the first rack 311 and the second rack 411 disclosed in the present embodiment is the stroke length that the adjusting gear 51 is able to adjust. The adjusting gear 51 meshes the first rack 311 and the second rack 411 to increase or decrease the length of the overlapping portions. Here, the length of the first rack 311 and the second rack 411 is not limited and can be set according to actual usage requirements.

The first connecting member 30 and the second connecting member 40 disclosed in the present embodiment are made of PP material, which has excellent softness and plastic strength. The first connecting member 30 and the second connecting member 40 can move toward each other while driven by the adjusting gear 51 to decrease the head circumference size, or move away from each other while driven by the adjusting gear 51 to increase the head circumference size.

It should be noted that, in the present embodiment, only the materials of the first connecting member 30 and the second connecting member 40 are exemplified, but the protection scope of the present disclosure is not limited thereto. Other types of materials that are able to achieve the technical effects disclosed in the present disclosure and are equivalent replacements for the concept of the present disclosure, should also be within the scope of protection of the present disclosure.

The first connecting member 30 and the second connecting member 40 disclosed in the present embodiment are arranged in an arc shape, and two free ends are connected to the head-worn device to form a ring shape surrounding the user's head. The first connecting member 30 and the second connecting member 40 are also arranged with hollow grooves, and the hollow grooves can reduce the weight of the first connecting member 30 and the second connecting member 40, thereby reducing the difficulty of wearing.

The first rack 311 and the second rack 411 disclosed in the present embodiment are arranged opposite to each other. In the present embodiment, as shown in FIG. 2, the first rack 311 is arranged on an upper side of the first strip-shaped through hole 31 and on a left side of the adjusting gear 51, and the second rack 411 is arranged on a lower side of the second strip-shaped through hole 41 and on a right side of the adjusting gear 51. The two racks are arranged relative to each other and mesh with the adjusting gear 51, that is, when the adjusting gear 51 rotates clockwise, the first connecting member 30 and the second connecting member 40 move toward each other; when the adjusting gear 51 rotates counterclockwise, the first connecting member 30 and the second connecting member 40 move away from each other to achieve tightness adjustment.

As shown in FIG. 4, FIG. 5 and FIG. 6, as another implementation of the present embodiment, a ratchet ring 1211 is disclosed to be arranged on a side wall of the mounting hole 121, and the knob 50 includes a mounting cover 52, which is arranged on a side of the mounting hole 121 away from the accommodating space. That is, the mounting cover 52 is arranged on the outside for the convenience of the user to touch and rotate. A central shaft 521 is arranged on a side of the mounting cover 52 facing the accommodating space, and the adjusting gear 51 is sleeved on the central shaft 521. The mounting cover 52 is used to drive the adjusting gear 51 to rotate, and through the mounting cover 52 the user can drive the central shaft 521 and the adjusting gear 51 sleeved on the central shaft 521 to rotate, so as to flexibly adjust the movement of the first connecting member 30 and the second connecting member 40.

The adjusting gear 51 disclosed in the present embodiment is fixedly connected to the mounting cover 52. The central shaft 521 is arranged with a support column protruding along its radial direction, the adjusting gear 51 is arranged with a central hole for inserting the central shaft 521, and a side wall of the central hole is arranged with a support groove adapted to the support column. The adjusting gear 51 and the mounting cover 52 are fixedly connected via the support column and the support groove. It should be noted that the fixed connection method between the adjusting gear 51 and the central shaft 521 is not limited to the present embodiment.

The knob 50 disclosed in the present embodiment further includes a stopping body 53, which is sleeved on the central shaft 521 and rotates synchronously with the adjusting gear 51. A pawl portion 531 is arranged on an outer peripheral wall of the stopping body 53. The pawl portion 531 includes a stopping surface 5311 and a guiding inclined surface 5312. When the stopping body 53 rotates in a direction toward the stopping surface 5311, the stopping surface 5311 engages with the ratchet ring 1211; when the stopping body 53 rotates in a direction toward the guiding inclined surface 5312, the guiding inclined surface 5312 and the ratchet ring 1211 slide relative to each other.

The stopping surface 5311 of the pawl portion 531 disclosed in the present embodiment engages with the ratchet ring 1211 to play a certain buffering role, which can limit the rotation of the central shaft 521, so that the adjusting gear 51 stays at a specific position. The user can conveniently fix the mounting cover 52 at a position with desired tightness to avoid the adjustment of the adjusting gear 51 due to accidental rotation during use, and prevent the first connecting member 30 and the second connecting member 40 from loosening by themselves. Even if the head-worn device is affected by movement or external force, the first connecting member 30 and the second connecting member 40 can be stably maintained at the position with desired tightness and are not prone to loosening or sliding.

The guiding inclined surface 5312 disclosed in the present embodiment is design to slide relative to the ratchet ring 1211 when the first connecting member 30 and the second connecting member 40 move toward each other, while the stopping surface 5311 and the ratchet ring 1211 are designed to be engaged with each other when the first connecting member 30 and the second connecting member 40 move away from each other, since the first connecting member 30 and the second connecting member 40 may loosen themselves, and can not tighten by themselves.

As another implementation of the present embodiment, an angle between the guiding inclined surface 5312 and the stopping body 53 is an acute angle. When the stopping body rotates toward the guiding inclined surface 5312, the angle between the guiding inclined surface 5312 and the stopping body being an acute angle would not lead to contact surfaces against each other. The ratchet ring 1211 does not limit the rotation of the stopping body 53 toward the guiding inclined surface 5312. An angle between the stopping surface 5311 and the stopping body 53 is a right angle or an obtuse angle. When the stopping body rotates toward the stopping surface 5311, contact surfaces between the stopping surface 5311 and the ratchet ring 1211 against each other, so that the pawl portion 531 is engaged with the ratchet ring 1211, thereby limiting the rotation of the stopping body 53.

Exemplarily, as shown in FIG. 5, when the first connecting member 30 and the second connecting member 40 loosen by themselves, the adjusting gear 51 is driven to rotate clockwise. At this time, due to the engagement of the stopping surface 5311 and the ratchet ring 1211, the adjusting gear 51 is prevented from rotating, that is, the first connecting member 30 and the second connecting member 40 are prevented from loosening by themselves.

As another implementation of the present embodiment, the stopping body 53 and the adjusting gear 51 are integrally formed. When the user adjusts the mounting cover 52, the stopping body 53 and the adjusting gear 51 rotate synchronously to more accurately adjust the tightness of the head circumference. The connection between the stopping body 53 and the adjusting gear 51 is firm, which reduces the possibility of loosening and swinging, and improves the assembly quality and service life.

The stopping body 53 disclosed in the present embodiment is integrally formed with the pawl portion 531, so that when the pawl portion 531 is engaged with the ratchet ring 1211, the pawl portion 531 is difficult to be damaged, thereby extending the service life of the pawl portion 531.

As another implementation of the present embodiment, at least two pawl portions 531 are arranged at intervals along a circumferential direction on an outer edge of the ratchet ring 1211. The arrangement of the two pawl portions 531 can provide better connection stability, increase the abutment area, and evenly share the bearing force of the connecting members. The two pawl portions 531 being arranged at intervals along circumferential direction can better resist the influence of external forces and reduce the risk of loosening of the first connecting member 30 and the second connecting member 40. The head-worn device is ensured to remain stable during use and is not prone to loosening or falling off.

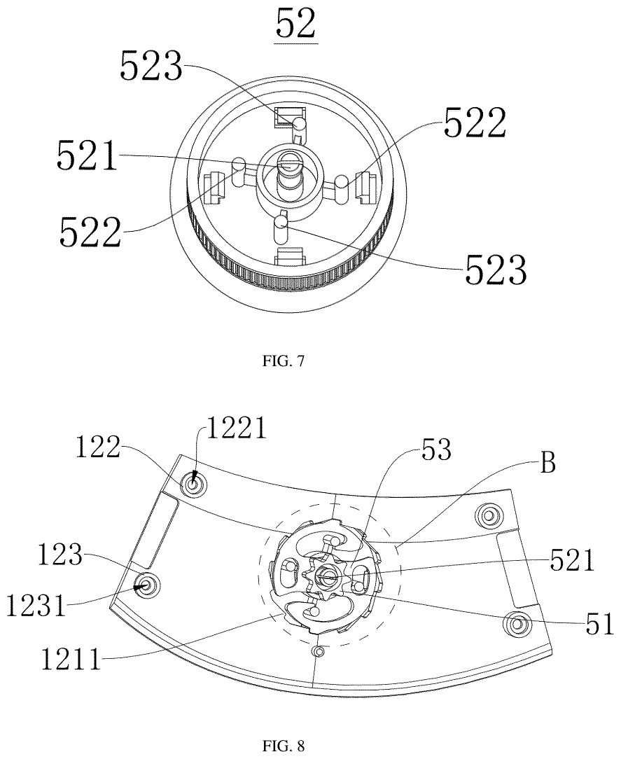

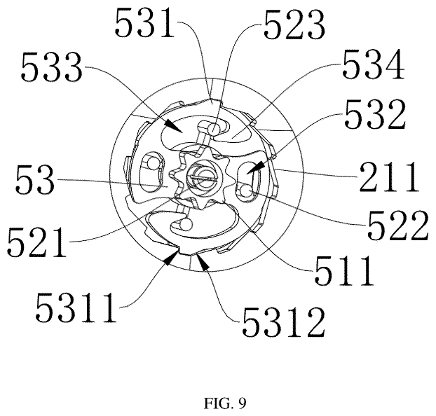

As shown in FIG. 7, FIG. 8 and FIG. 9, as another implementation of the present embodiment, a rib column 522 is arranged on one side of the mounting cover 52 facing the accommodating space, and an arc groove 532 adapted to the rib column 522 is arranged on the stopping body 53. The rib column 522 is inserted into the arc groove 532. When the mounting cover 52 rotates, the rib column 522 abuts against the arc groove 532 to push the stopping body 53 to rotate. The purpose of providing the rib column 522 is to enable the stopping body 53 to rotate when adjusting the mounting cover 52, and also to increase the connection mode between the mounting cover 52 and the stopping body 53, so that the structure is more compact and the connection is more firm.

Two rib columns 522 and two arc grooves 532 are disclosed in the present embodiment and the two rib columns 522 and the two arc grooves 532 are arranged correspond to each other. Through the positioning of the two rib columns 522 and the guidance of the two arc grooves 532, the accurate alignment of the components during the assembly process can be ensured. During the rotation of the mounting cover 52, the two rib columns 522 contact an inner wall of the arc grooves 532, pushing the stopping body 53 to rotate.

As another implementation of the present embodiment, a push column 523 is further arranged on the side of the mounting cover 52 facing the accommodating space, and a hook opening 533 is arranged on the stopping body 53. An end of the stopping body 53 is bent toward the hook opening 533 to form a hook-shaped part 534, and the push column 523 is inserted into the hook opening 533. When the stopping body 53 rotates in a direction toward the stopping surface 5311, the push column 523 pushes the hook-shaped part 534 inward to ensure the stopping surface 5311 is disengaged with the ratchet ring 1211.

Exemplarily, as shown in FIG. 9, when the stopping body 53 rotates in the clockwise direction, the push column 523 moves in a direction toward the hook-shaped part 534 and pushes the hook-shaped part 534, so that the hook-shaped part 534 is deformed toward an axis of the stopping body 53, thereby driving the pawl portion 531 on the stopping body 53 to also deform toward the axis of the stopping body 53, so that the pawl portion 531 is no longer engaged with the ratchet ring 1211 and is out of engagement. At this time, the first connecting member 30 and the second connecting member 40 can be adjusted to move toward or away from each other at will. When the mounting cover 52 is rotated to a suitable position, the rotational force is no longer applied to the stopping body 53, the push column 523 no longer pushes the hook-shaped part 534, and the pawl portion 531 resumes to be engaged with the ratchet ring 1211.

When rotating counterclockwise, the contact surface between the pawl portion 531 and the ratchet ring 1211 is an inclined surface, and the pawl portion 531 does not produce a blocking effect. Therefore, the length of the overlapping portion can be easily adjusted by rotating the mounting cover 52 with one hand, which is easy to operate.

Two push columns 523 and two hook openings 533 are disclosed in the present embodiment, which increase the reliability of the head-worn device. The hook-shaped part 534 and the pawl portion 531 are arranged on a same radial direction of the stopping body, so that when the hook-shaped part 534 is deformed, the pawl portion 531 is also deformed synchronously to release the engaging state.

The push columns 523 and the rib columns 522 disclosed in the present embodiment are arranged in an alternating sequence at intervals. The arrangement of the push columns 523 can also reduce the abutment force between the pawl portion 531 and the ratchet ring 1211, prevent the pawl portion 531 from breaking, and extend the service life of the pawl portion 531.

The stopping body 53 disclosed in the present embodiment is an elastic stopping body 53. When the pawl portion 531 is subjected to a large rotational force, the pawl portion 531 can produce elastic deformation to prevent the pawl portion 531 from breaking. By setting the knob 50, the number of hardware is reduced, which can reduce the weight of the head-worn device.

As shown in FIG. 5 and FIG. 8, as another implementation of the present embodiment, the hollow shell 10 includes a wearable shell 11 and an assembly shell 12, the assembly shell 12 cooperates with the wearable shell 11 to form the accommodating space. The headband 20 is arranged on a side wall of the wearable shell 11, and the mounting hole 121 is opened on the assembly shell 12. A first guiding column 122 and a second guiding column 123 are arranged on a side of the assembly shell 12 facing the accommodating space, the first guiding column 122 and the second guiding column 123 are arranged at intervals along a width direction of the assembly shell 12, and the first connecting member 30 or the second connecting member 40 is arranged between the first guiding column 122 and the second guiding column 123, that is, the first connecting member 30 or the second connecting member 40 is clamped between the first guiding column 122 and the second guiding column 123. Therefore, the first connecting member 30 and the second connecting member 40 are facilitated to move along a length direction of the assembly shell 12, thereby improving the efficiency of the first connecting member 30 and the second connecting member 40 moving toward or away from each other.

Further, a first positioning groove 1221 is arranged on the first guiding column 122 disclosed in the present embodiment, and a first positioning column 111 adapted to the first positioning groove 1221 is arranged on the wearable shell 11. The first positioning column 111 is inserted into the first positioning groove 1221. And/or, a second positioning groove 1231 is arranged on the second guiding column 123, and a second positioning column 112 adapted to the second positioning groove 1231 is arranged on the wearable shell 11. The second positioning column 112 is inserted into the second positioning groove 1231. Thus, the wearable shell 11 is fixedly connected to the assembly shell 12, thereby improving the overall reliability.

In summary, the present disclosure provides a head-worn device with adjustable tightness and easy operation, which is arranged with a hollow shell 10 for wearing on the top of the user's head, and is also arranged with a first connecting member 30, a second connecting member 40 and a headband 20. The headband 20 passes through through holes 110 and is connected to the first connecting member 30 and the second connecting member 40, so that the entire head-worn device can be tightly fixed on the user's head to reduce movement and shaking. The first connecting member 30 and the second connecting member 40, connected with the headband 20, form an annular structure that encircles the user's head. The user can adjust the relative movement of the first connecting member 30 and the second connecting member 40 toward or away from each other via a knob 50, thereby adjusting the length of the overlapping portion between the first connecting member 30 and the second connecting member 40. The adjustment can be performed without additional tools or operations, allowing for precise tightness adjustment to accommodate users with different head sizes. Furthermore, the adjustment can be performed by touch without requiring visual confirmation, making the operation simple and convenient. The present disclosure reduces sliding and shaking of the head-worn device, providing improved stability.

The disclosed head-worn device provides stability and comfort during long-term use. By using the head-worn device, the tightness of the head-worn device can be flexibly adjusted according to the head circumference of different users to obtain better wearing comfort and stability.

It should be noted that, in the absence of conflict, the embodiments and features in the embodiments of the present disclosure may be combined with each other.

It should be noted that the present disclosure takes a head-worn device as an example to introduce the structure and working principle of the present disclosure, but the present disclosure is not limited to the head-worn device, and can also be applied to the production and use of other similar products.

It should be understood that the present disclosure is not limited to the precise structure described above and shown in the drawings, and various modifications and substitutions can be made without departing from the protecting scope. The scope of the present disclosure is limited only by the appended claims.

The above description is only an embodiment of the present disclosure and is not intended to limit the present disclosure. Any modifications, equivalent substitutions, improvements, etc. made within the spirit and principles of the present disclosure shall be included in the protection scope of the present disclosure.

What is claimed is:

1. A head-worn device, comprising:
a hollow shell, an accommodating space is formed in the hollow shell, and two through holes are oppositely arranged on an inner side wall of the hollow shell; a mounting hole is arranged on an outer side wall of the hollow shell; wherein the mounting hole and the through holes are all connected to the accommodating space;
a first connecting member and a second connecting member, the first connecting member and the second connecting member are arranged in parallel in the accommodating space, and the first connecting member and the second connecting member are both able to reciprocate along an axial direction of the accommodating space;
a knob, the knob is inserted into the mounting hole and extends between the first connecting member and the second connecting member; two sides of the knob are respectively connected to the first connecting member and the second connecting member, and are configured to drive the first connecting member and the second connecting member to move toward or away from each other; and
a headband, the headband is arranged on a side wall of the hollow shell; two ends of the headband respectively pass through the two through holes and are respectively connected to the first connecting member and the second connecting member.

2. The head-worn device according to claim 1, wherein a first strip-shaped through hole is arranged on the first connecting member, and a first rack is arranged on a side wall of the first strip-shaped through hole along a length direction; a second strip-shaped through hole is arranged on the second connecting member, and a second rack is arranged on a side wall of the second strip-shaped through hole along a length direction;
wherein the first rack and the second rack are arranged opposite to each other, and the knob comprises an adjusting gear, the adjusting gear is rotatably arranged in the first strip-shaped through hole and the second strip-shaped through hole, a gear ring portion is arranged on a circumference of the adjusting gear, and the first rack and the second rack are respectively meshed with opposite sides of the gear ring portion.

3. The head-worn device according to claim 2, wherein a ratchet ring is arranged on a side wall of the mounting hole, and the knob comprises:
a mounting cover, the mounting cover is arranged on one side of the mounting hole away from the accommodating space, a central shaft is arranged on the mounting cover, and the adjusting gear is sleeved on the central shaft, the mounting cover is configured to drive the adjusting gear to rotate; and
a stopping body, the stopping body is sleeved on the central shaft and rotates synchronously with the adjusting gear, a pawl portion is arranged on an outer peripheral wall of the stopping body, the pawl portion comprises a stopping surface and a guiding inclined surface, and when the stopping body rotates in a direction toward the stopping surface, the stopping surface engages with the ratchet ring; when the stopping body rotates in a direction toward the guiding inclined surface, the guiding inclined surface and the ratchet ring slide relative to each other.

4. The head-worn device according to claim 3, wherein an angle between the guiding inclined surface and the stopping body is an acute angle; and
an angle between the stopping surface and the stopping body is a right angle or an obtuse angle.

5. The head-worn device according to claim 3, wherein a rib column is arranged on a side of the mounting cover facing the accommodating space, and an arc groove adapted to the rib column is arranged on the stopping body, the rib column is inserted into the arc groove, and when the mounting cover rotates, the rib column abuts against the arc groove to push the stopping body to rotate.

6. The head-worn device according to claim 3, wherein a push column is arranged on a side of the mounting cover facing the accommodating space, a hook opening is arranged on the stopping body, an end of the stopping body is bent toward the hook opening to form a hook-shaped part, and the push column is arranged to pass through the hook opening, when the stopping body rotates in a direction toward the stopping surface, the push column pushes the hook-shaped part inward to disengage the stopping surface and the ratchet ring.

7. The head-worn device according to claim 3, wherein the stopping body and the adjusting gear are integrally formed; and/or
the stopping body and the pawl portion are integrally formed.

8. The head-worn device according to claim 4, wherein at least two pawl portions are arranged at intervals along a circumferential direction on an outer edge of the ratchet ring.

9. The head-worn device according to claim 1, wherein the hollow shell comprises a wearable shell and an assembly shell, the assembly shell cooperates with the wearable shell to form the accommodating space, the headband is arranged on the wearable shell, and the mounting hole is opened on the assembly shell;
wherein a first guiding column and a second guiding column are arranged on a side of the assembly shell facing the accommodating space, the first guiding column and the second guiding column are arranged at intervals along a width direction of the assembly shell, and the first connecting member or the second connecting member is arranged between the first guiding column and the second guiding column.

10. The head-worn device according to claim 9, wherein a first positioning groove is arranged on the first guiding column, a first positioning column adapted to the first positioning groove is arranged on the wearable shell, and the first positioning column is inserted into the first positioning groove; and/or
a second positioning groove is arranged on the second guiding column, a second positioning column adapted to the second positioning groove is arranged on the wearable shell, and the second positioning column is inserted into the second positioning groove.
